(19) United States
(12) Patent Application Publication
(10) Pub. No.: US 2009/0012326 A1
(43) Pub. Date: Jan. 8, 2009

(54) Use of Salicylate as an Antidote for Paraquat Intoxications in Mammals

(75) Inventors: Felix Dias Carvalho, Santo Tirso (PT); Ricardo Jorge Dinis Oliveira, Santo Tirso (PT); Maria de Lourdes Pinho De Almeida Souteiro Bastos, Porto (PT); Fernando Manuel Gomes Remiao, Pedroso (PT); Jose Alberto Ramos Duarte, Porto (PT); Amparo Sanchez Navarro, Salamanca (ES)

Correspondence Address: SUGHRUE MION, PLLC, 2100 PENNSYLVANIA AVENUE, N.W., SUITE 800, WASHINGTON, DC, 20037, US

(73) Assignee: UNIVERSIDADE DO PORTO, PORTO (PT)

(21) Appl. No.: 12/159,714

(22) PCT Filed: May 11, 2007
(86) PCT No.: PCT/IB2007/051799

§ 371 Date: Jun. 30, 2008

(30) Foreign Application Priority Data
May 12, 2006 (PT) 103480

(51) Int. Cl. C07C 65/10

(52) U.S. Cl. 562/477

(57) ABSTRACT

The present invention concerns to the use of salicylate in the treatment of mammal intoxications caused by the herbicide paraquat (PQ). It was achieved, for the first time, 100% of survival 30 days after the administration to Wistar rats, by intraperitoneal route, of a PQ dose that, in the absence of treatment, is itself 100% lethal at the end of 6 days. The administration of salicylate, two hours after PQ, reversed the PQ toxicity and extended the life of the animals to the levels of the control group.

### TECHNIQUE DOMAIN OF THE INVENTION

The present invention concerns to the use of salicylate in the treatment of mammal intoxications caused by the herbicide paraquat (PQ) (FIG. 1 e 2). It was achieved, for the first time, 100% of survival 30 days after the intraperitoneal administration, to Wistar rats, of a PQ dose that, in the absence of treatment, is itself 100% lethal at the end of 6 days. The administration of salicylate, two hours after PQ, reversed the PQ toxicity and extended the life of the animals to the levels of the control group, constituting this invention an important step in the fight against PQ intoxications.

### BACKGROUND OF THE INVENTION

Paraquat dichloride (methyl viologen; PQ) is an effective and widely used herbicide, known to cause thousands of deaths in mammals, from both accidental and voluntary ingestion, as well as from dermal exposure. The lethality is mainly the consequence of a severe pulmonary toxicity with the consequent respiratory failure. The extreme severity of this intoxication for which there is no available antidote, impelled the studies that conducted to this invention.

Since its introduction in agriculture in 1962 [1], the paraquat [(PQ: 1,1′-dimethyl-4,4′-bipyridynium ion: methyl viologen) FIG. 3], used as desiccant and defoliant in a variety of crops has caused thousands of deaths from both accidental and voluntary ingestion, as well as from dermal exposure [2-5]. Although it may be considered as one of the most toxic poisons frequently used for suicide attempts, it is readily available without restriction in several countries where it is registered. Depending on the ingested dose, different clinical patterns and outcomes have been observed in experimental animals and humans. PQ accumulates preferably in the lung through a transport system for which the polyamines are the natural substrates [1]. In comparison to other organs, the lungs, and more specifically the alveolar epithelial and Clara cells, are endowed with a particularly active polyamine uptake system [6-10]. This is the main reason for the lung to constitute the target organ for PQ toxicity [1].

Briefly, the direct cellular toxicity of PQ is essentially due to its redox cycling (FIG. 2): PQ is reduced enzymatically, mainly by NADPH-cytochrome P-450 reductase [11] and NADH:ubiquinone oxidoreductase (complex I) [12, 13], to form the PQ monocation free radical (PQ.⁺). The PQ.⁺ is then rapidly re-oxidized in the presence of oxygen (which exhibits high concentrations in the lung), thus resulting in the generation of the superoxide radical (O₂.⁻) [14, 15]. This then sets in the well-known cascade leading to generation of reactive oxygen species (ROS), namely the hydrogen peroxide and the hydroxyl radical (HO.) with the consequent deleterious effects characteristics of oxidative stress. Indeed, hydroxyl radicals [16, 17] have been implicated in the initiation membrane-damage by lipid peroxidation during the exposure to paraquat in vitro [1,6] as well as in vivo [15, 18] by attack on polyunsaturated lipids, depolymerization of hyaluronic acid, inactivation of proteins and damage of DNA.

Nowadays, no antidote or effective treatment for PQ poisoning has been identified, the survival being mainly dependent on the amount ingested and the time elapsed until the patient is submitted to intensive medical measures to inactivate or to eliminate PQ, before its absorption and/or cellular uptake. These approaches, are aimed to prevent PQ accumulation in the tissues, specially in the lungs, and include procedures such as induction of emesis and/or intestinal transit, gastric lavage, administration of oral adsorbents, hemodialysis and hemoperfusion [19-23].

Besides these treatments, additional protective measures have also been adopted: (i) to prevent the generation of ROS, namely the effective iron chelation by desferrioxamine [24, 25]; (ii) to scavenge ROS including the maintenance of effective levels of antioxidants, such as vitamin-E [26]; (iii) those aimed to repair the ROS-induced lesions, particularly the maintenance of effective levels of reduced glutathione (GSH) by administrating N-acetylcysteine [27, 28], and (iv) those aimed to reduce inflammation (normally administered few days after intoxication) by administration of dexamethasone (DX) [29, 30], methylprednisolone (MP) [31]cyclophosphamide (CP) [31] and NAC [27, 28].

However, such treatments are mainly palliative and the mortality rate remains above 70%, even when multiple therapies are concomitantly administered. Our research group obtained an increase of the elimination of PQ accumulated in the lung through the induction of de novo synthesis of P-glycoprotein (P-gp). The administration of an inductor of P-gp de novo synthesis, 2 hours after PQ exposure of rats, led to a remarkable decrease of PQ accumulation in the lung, to less than 40% of the group exposed only to PQ (control group), and to an increase of its faecal excretion with the subsequent reversion of several biochemical and histopathological biomarkers of toxicity in just 24 hours. The survival increased to 50% at the end of 10 days in opposition to 10% reported in previous studies with the same time study duration [32] and to other studies where the evaluated time was only 3 days [33]. Such invention, constituted a landmark in toxicology and an important step in the fight against PQ and several other xenobiotic intoxications.

### DESCRIPTION OF THE INVENTION

In spite of our proposed antidotal pathway, through the induction of de novo synthesis of P-glycoprotein (P-gp), to be used in the treatment of mammals PQ poisonings, the persistent gap related to the inexistence of an antidote that conducts to 100% of survival, impelled the study herein described. It is objective of the present invention to provide an effective solution to reduce the toxicity of PQ in the lung, increasing the percentage of success of the treatments and consequently the survival of the intoxicated mammals by PQ. The present invention concerns to the use of the drug—sodium salicylate (FIG. 5)—in the treatment of mammals PQ intoxications (FIG. 3).

The Aspirin® (acetylsalicylic acid) is one of the most widely used medicines with an average annual consumption of about 30 g per person in industrialized countries [34]. In the United States alone, 35,000 kg Aspirin® is consumed daily [35]. Ingested acetylsalicylic acid mainly is absorbed as such, but some enters the systemic circulation as salicylic acid after hydrolysis by esterases in the gastrointestinal mucosa and liver. Acetylsalicylic acid can be detected in the plasma only for a short time as a result of hydrolysis in plasma, liver, and erythrocytes. In fact, 30 minutes after a dose of 0.65 g, only 27% of the total plasma salicylate is in the acetylated form. Due to this rapid deacetylation, it has been assumed that anti-inflammatory effects of Aspirin® are largely mediated by salicylate [36]. This assumption receives support by experimental evidence that in vivo, salicylate and acetylsalicylic acid exhibit similar anti-inflammatory potentials [37], eventhough, the salicylates are not able to acetylate the serine residue of the active cyclooxygenase site, as occurs with Aspirin® [38, 39]. In addition, plasma half-life of acetylsalicylic acid is about 15 min whereas for salicylate is between 2 and 30 hours depending on the administered dosage. Concerning to distribution, more than 80% of circulating salicylate is bound to plasma proteins, specially to albumin [40], showing also the tendency to accumulate in inflamed tissues [41]. Importantly, the sodium salicylate has been used in the treatment of rheumatic diseases for more than 130 years [42].

So far, several mechanisms have been implicated in the salicylate anti-inflammatory effects, including (i) cyclooxygenase inhibition [43, 44], (ii) p38 mitogen-activated protein kinase (MAPK) activation [45], (iii) Nuclear Factor (NF)-κB inhibition [46], (iv) peroxisome proliferator-activated receptor δ inhibition [47] and (v) antioxidant properties by trapping HO. Radicals [48]. With the exception of reported salicylate-induced activation of p38 MAPK [45], observed effects are usually inhibitory. The transcription factor, NF-κB, has been regarded as a key element in the response of cells to inflammatory stimuli. NF-κB activity is attributed to the Rel/NF-κB family proteins forming homo- and heterodimers through the combination of the subunits p65 (or RelA), p50, p52, c-Rel and RelB, which bind to DNA target sites, where they directly regulate gene transcription. In most cells, NF-κB (the designation for p50-RelA, the heterodimer most frequent) is retained in the cytoplasm as an inactive complex bound to inhibitory proteins (IκB; IκBα, -β, -ε, -γ, p105, p100 and Bcl-3; for revision see [49, 50]. Bacterial lipopolysaccharides (LPS), IL-1β, tumor necrosis factor (TNF)-α, UV light, ROS and double-stranded RNA are classical inducers of NF-κB. They induce intracellular signaling cascades that activate the IκB kinases (IKKs) that phosphorylate serines 32 and 36 of IκBα, leading IκBα to polyubiquitination which directs this inhibitory protein to degradation by the 26S proteossome, and consecutive activation of NF-κB [51]. When IκBα is degraded, NF-κB migrates to the nucleus, where it binds to the KB-sites in the promoter region of target genes and regulates their transcription. Targets include pro-inflammatory enzymes, cytokines, chemokines, apoptosis inhibitors, cell adhesion molecules, the IκBα gene and many others. The anti-inflammatory effect of Aspirin® and salicylate has been linked to the inhibition of the NF-κB pathway, as shown by several studies [52-55].

Since ROS are classical inductors of NF-κB and PQ-induced inflammation is mediated by the production of ROS generated by the redox-cycle described above, with this invention we aimed to corroborate the NF-κB involvement in lung toxicity as consequence of PQ-acute exposure and in a second step to inhibit this signaling pathway on its first steps. For this approach we used sodium salicylate, which has been shown to inhibit the NF-κB pathway by impeding IκB phosphorylation [55]. Indeed, a single dose of sodium salicylate, reversed the PQ toxicity and, more significantly, it was achieved a survival of 100% of rats 30 days after PQ-exposure. Sodium salicylate constitutes the first real PQ antidote described with such degree of success.

With the industrial application of the invention herein described, it will be given an important step in the treatment of PQ intoxications. Importantly, the described invention resulted from the administration of sodium salicylate, 2 hours after the intoxication with PQ of animals. This period of time confers more realism in the application in human intoxications, since it reflects, in the majority of the cases, the time that the intoxicated patient takes to arrive to the hospital emergency room. The novelty concerning to this invention is that the use of sodium salicylate in the treatment of intoxications by PQ of mammals proved to be able to originate, for lethal doses of this herbicide, a survival of 100%.

### BRIEF DESCRIPTION OF THE FIGURES

FIG. 1. Percentage of Rats survival, in the control, paraquat (PQ) and paraquat plus sodium salicylate (PQ+NaSAL) groups. ^{ccc}p<0.001 versus PQ group.

FIG. 2. Effect of treatment in the weight of Rats, from the control, paraquat (PQ) and paraquat plus sodium salicylate (PQ+NaSAL) groups. cccp<0.001 versus PQ group.

FIG. 3. Chemical structure of PQ and its salts available in the market.

FIG. 4. Schematic representation of the mechanism of PQ toxicity. A. Cellular diaphorases, SOD. Superoxide dismutase or spontaneously, CAT. Catalase, Gpx. Glutathione Peroxidase, Gred. Glutathione Reductase, PQ²⁺. Paraquat, PQ⁺. Paraquat cation free radical, FR. Fenton reaction, HWR. Haber-Weiss Reaction.

FIG. 5. Chemical structure of sodium salicylate.

FIG. 6.I Light (A) and electron (B) micrographs of the lung from animals of control group, showing (in A and B) a normal pulmonary structure without evidences of alveolar collapse, vascular congestion or cellular infiltrations; in B are observed pneumocytes Type I and II (original magnification: A—640×; B—1.600×).

FIG. 6.II Light (A) and electron (B) micrographs of the lung from animals of the sodium salicylate 96 h group (200 mg/Kg, i.p.), showing a preserved structure with the presence of dispersed phagocytes; in B are observed two pneumocytes Type II and one macrophage (original magnification: A—1.050×; B—4.000×).

FIG. 6.III Light (A) and electron (B) micrographs of the lung tissue from animals of the paraquat 24 h group (25 mg/Kg, i.p.), showing a marked vascular congestion and a notorious generalized alveolar collapse (in A); beyond the mitochondrial swelling of endothelial cells, it is possible to observe (in B) an intravascular clot of platelets, with evident signs of activation, suggesting a vascular obstruction (original magnification: A—640×; B—10.000×).

FIG. 6.IV Light (A) and electron (B) micrographs of the lung tissue from animals of the paraquat+sodium salicylate 24 h group, showing (in A) a light alveolar collapse with whitening of its walls, as well as the presence of few phagocytes within capillaries; in B, beyond hypodense regions, suggestive of edema, and mitochondrial swelling affecting mainly the pneumocytes Type I, are also notorious two macrophages within alveolus (original magnification: A—900×; B—2.500×).

FIG. 6.V Light (A) and electron (B) micrographs of the lung tissue from animals of the paraquat 48 h group, showing (in A) areas of necrosis of the alveolar wall and macrophages, cellular debris and inflammatory infiltrate within the alveolar space. It is also observed capillaries filled with angular erythrocytes, suggestive of vascular stasis; in B it is possible to observe three blood vessels obstructed by platelets with notorious sighs of activation and interstitial hypodense areas suggestive of edema (original magnification A—900×; B—4.000×).

FIG. 6.VI Light (A) and electron (B) micrographs of the lung tissue from animals of the paraquat+sodium salicylate 48 h group, showing (in A) an apparent preserved alveolar structure but with debris and phagocytes within the alveolar space and the presence of cytoplasmatic inclusions in some phagocytes; in B, beyond the intravascular polymorphonuclear, it is also observed cellular debris in the alveolar space as well as the presence of one macrophage and fibroblasts and collagen fibres within the interstitial space (original magnification: A—900×; B—3.150×).

FIG. 6.VII Light (A) and electron (B) micrographs of the lung from animals of the paraquat 96 h group, showing (in A) an intense vascular congestion as well as the presence of cellular debris and phagocytes within the alveolar space (in A); in B is noteworthy signs of fibroblastic activation with the presence of wide areas of the interstitial space filled with collagen fibres (original magnification: A—640×; B—6.300×).

FIG. 6.VIII Light (A) and electron (B) micrographs of the lung from animals of the paraquat+sodium salicylate 96 h group, showing (in A) a preserved alveolar structure, a few areas of necrosis of the alveolar wall and debris within the alveolar space, as well as the presence of alveolar and interstitial phagocytes with cytoplasmatic inclusions; in B it is observed two leukocytes in the intravascular space, one macrophage with cytoplasmatic inclusions similar to myelin figures and the existence of fibroblasts and collagen fibres in the interstitium (original magnification: A—900×; B—2.500×).

FIG. 7. Time-course of NF-κB activation induced by paraquat in lungs. Gel obtained by Fluorescence Electrophoretic Mobility Shift Assay (fEMSA). Lung tissue nuclear extracts from the different groups were prepared and subjected to fEMSA as described in Materials and Methods. Lane 1—control group; Lane 2—PQ 24 hours; Lane 3—PQ 48 hours; Lane 4—PQ 96 hours; Lane 5—blank; Lane 6—competition experiment with a 50-fold molar excess of a non-specific competitor (UC) in relation to specific probe (SP); Lane 7—competition experiment with a 50-fold molar excess of a specific competitor (SC, unlabelled specific probe) in relation to SP. Lane 8, 9 and 10-sodium salicylate 24, 48 and 96 h groups, respectively; Lane 11, 12 and 13—paraquat plus sodium salicylate 24, 48 and 96 h groups, respectively; The positions of specific NF-κB/DNA binding complexes (bands 1-3) are indicated. NS Band represents a no specific binding. The localization of the free probe (FP) is also indicated. The result presented is representative of three independent experiments, performed separately.

### DETAILED DESCRIPTION OF THE INVENTION

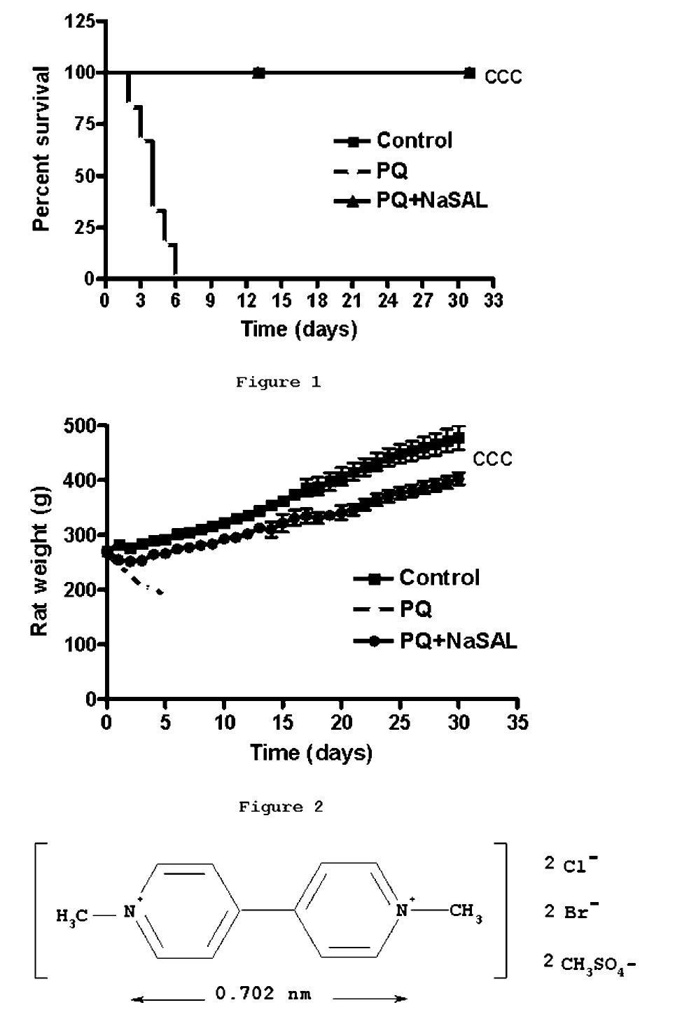

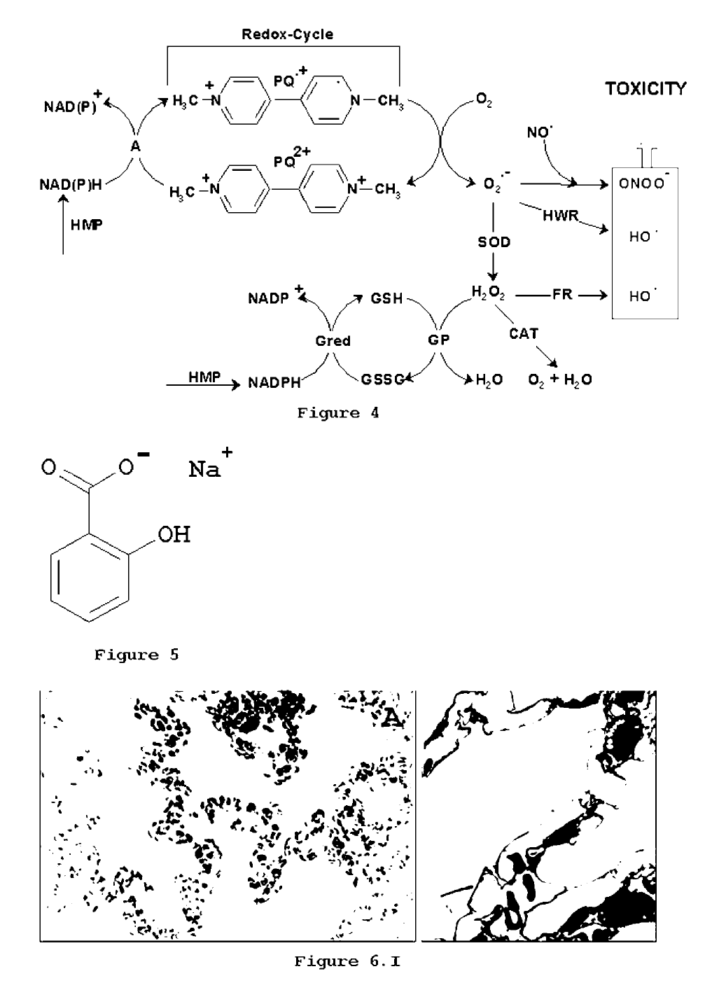

The present invention concerns to the use of salicylate in the treatment of mammal intoxications caused by the herbicide paraquat PQ (FIG. 1 e 2). The salicylate and derivatives, including Aspirin®, belongs to a class of easily available, inexpensive and widely used medicines that are collectively known as the non-steroidal anti-inflammatory drugs (NSAIDs). During more than two decades, the anti-inflammatory properties of those medicines containing this drug (such as Aspirin®), were exclusively attributed to the inhibition of prostaglandins and thromboxanes through the inhibition of cyclooxygenase activity [39]. Nowadays several mechanisms are attributed to salicylates contributing for its anti-inflammatory activities, including: (i) cyclooxygenase inhibition [43, 44], (ii) p38 mitogen-activated protein kinase (MAPK) activation [45], (iii) Nuclear Factor (NF)-κB inhibition [46], (iv) peroxisome proliferator-activated receptor δ inhibition [47] and (v) antioxidant properties by trapping HO. Radicals [48].

The great advantage of this invention was to obtain, for the first time, a complete reversion of PQ (25 mg/Kg, i.p.) toxicity and consequently the lethality through the administration of a potent anti-inflammatory drug—the sodium salicylate (200 mg/Kg, i.p.)—and by this way an increase of the survival of the animals to 100%.

No other previous treatment proved the achievement of a survival rate of 100%. As referred above, the toxicity of PQ depends on its lung accumulation where PQ leads to an oxidative stress condition by continuous ROS generation with subsequent inflammatory process and cellular damage. It is plausible to consider that this protection is of particular importance in the lung (by the raised points describe above), either by modification of PQ molecule and therefore abolishing its pulmonary capture or its capacity to generate redox-cycle.

The sodium salicylate (200 mg/kg of body weight, i.p.) was administered two hours after rats being intoxicated with PQ (25 mg/Kg of body weight). This time period between PQ exposure and sodium salicylate administration was established taking into account the time that normally occurs in the human reality between ingestion and the arrival of the intoxicated patient to the Emergency Services to initiate the therapy. The experimental dose of NaSAL was chosen according to numerous described studies to inhibit the NF-κB activation in vivo [56, 57].

Consequently, it was possible to reverse the pulmonary PQ toxicity, constituting this invention an important step against PQ mammal intoxications.

Below it is described one example of a way to concretize the invention using as a mammal model, the rat.

Reagents and Drugs

Paraquat, (methylviologen; 1,1′-dimethyl-4,4′-bipyridinium dichloride) and sodium salicylate (2-hydroxybenzoic acid sodium salt) were all obtained from Sigma (St. Louis, Mo., U.S.A.). The saline solution (NaCl 0.9%) and the sodium thiopental (0.5 g) were purchased from B. Braun (Lisbon, Portugal). All other reagents used were of analytical grade or of the highest grade available.

Treatment of Animals—In Vivo Studies

The study was performed using (n=84) adults male Wistar rats obtained from Charles River S. A. (Barcelona, Spain), with a mean weight of 250±10 g. Animals were randomly distributed in number of two per polypropylene cages with a stainless steel net at the top and wood chips at the screen bottom, in a air-conditioned room (alternate cycles of 12 hours light and darkness, room temperature 22±2° C. and with a relative humidity of 50-60%) at least one week (quarantine) before starting the experience in order to acclimate to their surrounding conditions. Animals were allowed access to tap water and rat chow ad libitum during the quarantine period. Housing and experimental treatment of animals were in accordance with National Institutes of Health guidelines (ILAR 1996). The experiments complied with the current laws of Portugal.

The biochemical and histological studies were carried out with 60 animals, randomly distributed to ten groups. Each animal was individually housed during the experimental period in a polypropylene cage with a stainless steel net at the top and wood chips at the screen bottom. Tap water and rat chow were given ad libitum during the entire experiment. The administrations of vehicle (0.9% NaCl), PQ, and sodium salicylate were all made intraperitoneally (i.p.) in an injection volume of 0.5 mL. Each group was treated as follows: (doses were given per Kg/body weight):

(I) Control group, n=6: animals treated with 0.9% NaCl. Animals were treated with one more administration of 0.9% NaCl two hours later and sacrificed 24 hours after the second injection;

(II) NaSAL group, n=18: animals treated with 0.9% NaCl. Animals were treated with one administration of NaSAL (200 mg/kg) two hours later and sacrificed 24 hours (n=6, NaSAL 24 h group), 48 hours (n=6, NaSAL 48 h group) and 96 hours (n=6, NaSAL 96 h group) after the second injection;

(III) PQ group, n=18: animals intoxicated with PQ (25 mg/kg). Animals were treated with one more administration of 0.9% NaCl two hours later and sacrificed 24 hours (n=6, PQ 24 h group), 48 hours (n=6, PQ 48 h group) and 96 hours (n=6, PQ 96 h group) after the second injection;

(IV) PQ+NaSAL group, n=18: animals intoxicated with PQ (25 mg/kg). Two hours later, animals were treated with NaSAL (200 mg/kg) and sacrificed 24 hours (n=6, PQ+NaSAL 24 h group), 48 hours (n=6, PQ+NaSAL 48 h group) and 96 hours (n=6, PQ+NaSAL 96 h group) after the second injection.

For the evaluation of survival rate, 24 animals were randomly divided into four groups (control, NaSAL, PQ and PQ+NaSAL) of six animals each. Animals were treated and kept under the same conditions as described above.

Treatments in all groups were always conducted between 8:00 and 10:00 a.m.

Tissue Processing for Histological Analysis

Two animals of each group were assigned to histological analysis. Lung samples were subjected to routine procedures for light microscopy (LM) and transmission electron microscopy (TEM) analysis. With the animals under anaesthesia, lung fixation was initiated in situ by perfusion through pulmonary artery, with 2.5% glutaraldehyde in 0.2 M sodium cacodylate buffer (pH 7.2-7.4) during 3 min. Subsequently, lungs were excised and sectioned into approximately 1 mm³ cubic pieces. After two washing steps, of 30 minutes each with buffer solution, the specimens were dehydrated in graded alcohol for 2 hours, and then embedded in Epon resin. Propylene oxide was the compound used in the dehydration-impregnation transition. The inclusion phase lasted 2 days. All the procedures were done at 4° C., with exception of the inclusion phase, which was performed at 60° C. Subsequent to the resin polymerization, semi-thin sections (thickening 1 μm) and ultra-thin sections (500 Å of thickness) were prepared (Ultracut, Leica), respectively for LM and TEM analysis. The grids, mounted with the ultra-thin specimens sections, were double-contrasted with 0.5% saturated uranyl acetateaqueoussolution during 30 min and then with 0.2% lead citrate solution for 15 min. The slides, mounted with semi-thin sections, were stained with toluidine blue. Five slides and three grids from each animal (standing ten slides and six grids per group), were examined in a Zeiss Phomi III photomicroscope and in a transmission electronic microscope (Zeiss EM 10A).

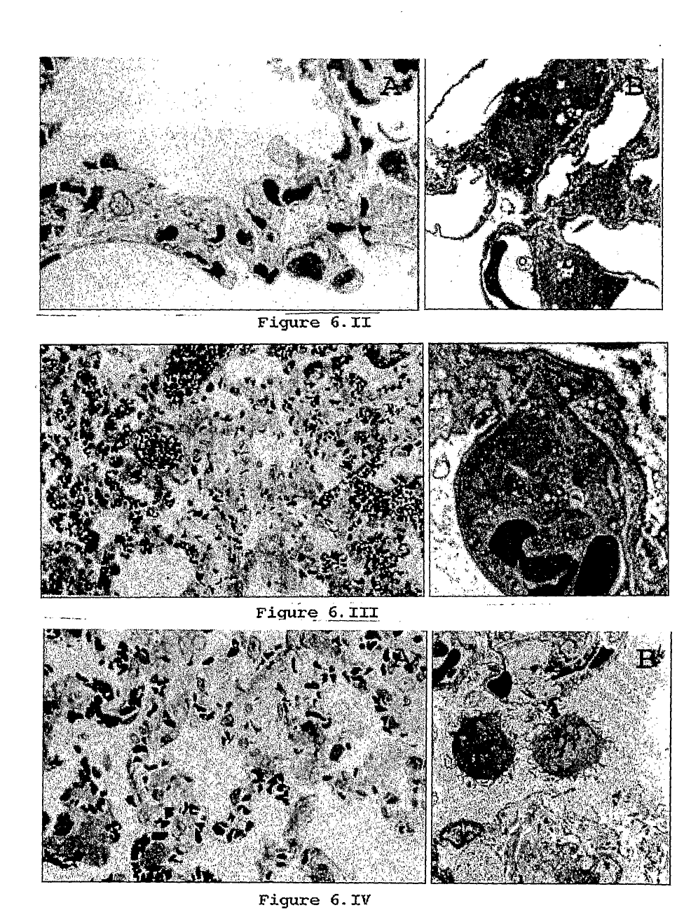

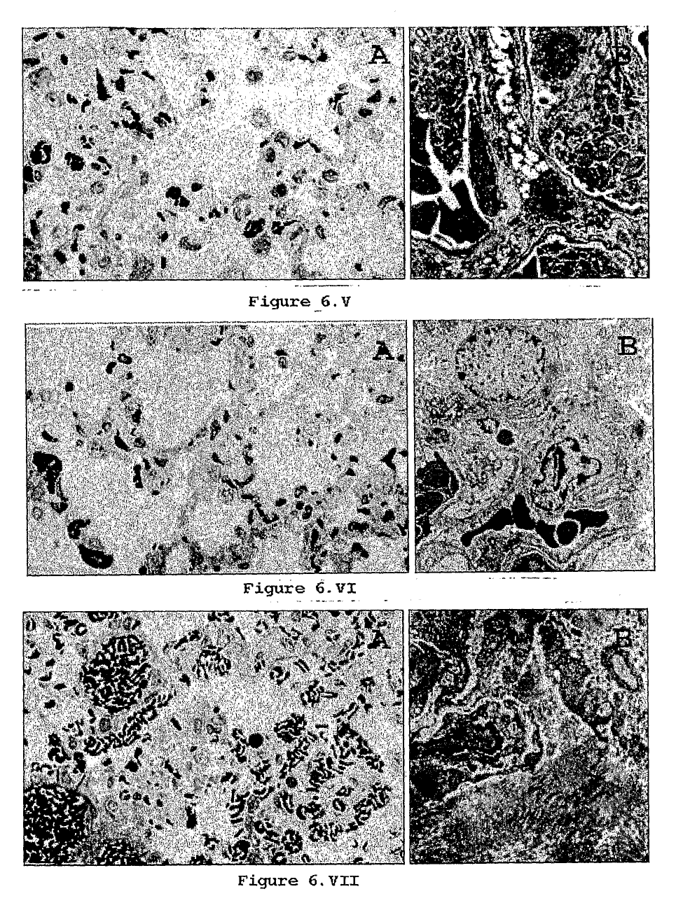

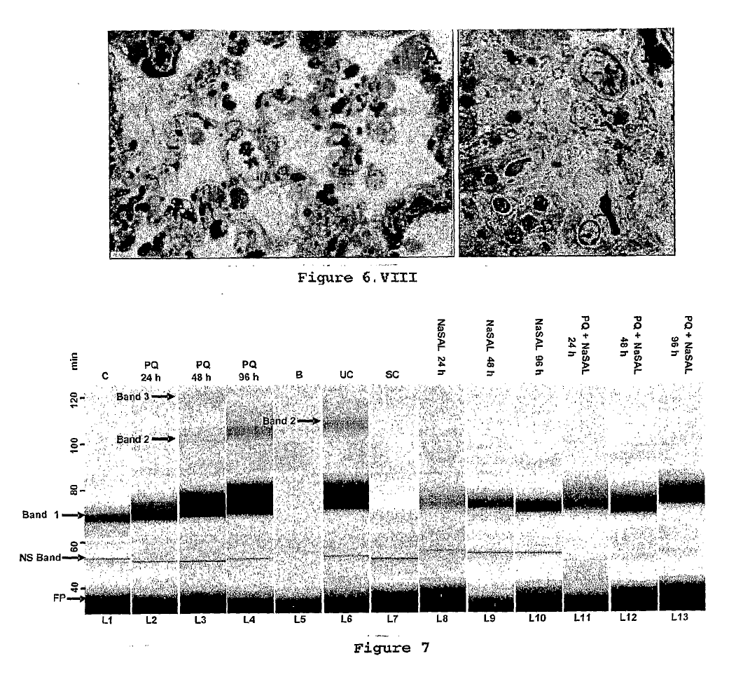

The evaluation of structural and ultrastructural analysis alterations was performed and registered those deviant relatively to control group (FIGS. 6.I-6.VIII).

Samples Processing for the Preparation of Nuclear Lung Extracts

Lungs were briefly homogenized (Ultra-Turrax® Homogenizer) in a AC buffer [(cell lysis buffer), 1 g of tissue/3 ml] containing: 10 mM Hepes (pH 7.9), 10 mM KCl, 1.5 mM MgCl₂, 0.2% Igepal, 0.5 mM EDTA, 0.1 mM EGTA, 1 mM dithiothreitol (DTT) and 0.25 mM phenylmethylsulfonyl fluoride (PMSF) and incubated on ice for 15 min. After a brief vortexing, the lysates were centrifuged (850 g, 4° C. for 10 min). The supernatants (cytoplasmic extracts) were discharged and the pellets were resuspended (washing step) in 500 μl of AC buffer and incubated for 15 min on ice and then centrifuged (14,000 g, 4° C., for 30 seconds). The supernatants (cytoplasmic extracts) were discharged and the pellets were resuspended in 500 μl of BC buffer (nuclei lysis buffer) containing: 20 mM Hepes, pH 7.9, 420 mM NaCl, 1.5 mM MgCl₂, 2% Igepal, 0.5 mM EDTA, 20% glycerol, 1 mM DTT, 0.25 mM PMSF, aprotinin (5 μg/ml), pepsatin (5 μg/ml), leupeptin (5 μg/ml) and incubated on ice for 30 min. After a brief vortexing, the lysates were centrifuged (14,000 g, 4° C. for 10 min). Supernatants (nuclear extracts) were collected, divided into aliquots and stored at −80° C. for posterior NF-κB semi-quantification by fEMSA. Protein quantification of the nuclear extracts was performed according to the method of Lowry et al. [58], using bovine serum albumin as standard.

Oligonucleotides

The synthetic oligonucleotides were purchased from Amersham Pharmacia Biotech (Uppsala, Sweden) and are summarized in Table 1.

Table 1—Oligonucleotides used in the study. Cy5 (indodicarbocyanine) is a fluorescence dye attached at 5′OH end of the oligonucleotide.

### [Table 1]

TABLE 1
[Table]

| | |
|---|---|
| Oligonucleotides | Sequence |

| | |
|---|---|
| NF-kB-FW-Cy5 | 5′-Cy5-GCC TGG GAA AGT CCC CTC AAC T-3′ |
| NF-kB-FW | 5′-GCC TGG GAA AGT CCC CTC AAC T-3′ |
| NF-kB-R | 5′-AGT TGA GGG GAC TTT CCC AGG C-3′ |

Determination of Transcriptional Activation of Lung Nuclear Proteins by Fluorescent Electrophoretic Mobility Shift Assay (fEMSA)

The NF-κB binding assay was performed according to a previously reported method [59]. Nuclear extracts (20 μg of protein) were incubated (one hour at 4° C.) in a fresh polypropylene tube with the following mixture: 0.5 pmol of specify double-stranded Cy5-labelled for each transcription factor, DNA-binding buffer [10 mM HEPES (pH 7.9), 0.2 mM EDTA, 50 mM KCl], 2.5 mM of DTT, 250 ng of poly(dI-dC). poly(dI-dC), 1% of Igepal and 10% glycerol; 9 μl of the mixture were resolved by electrophoresis on a 5% nondenaturing polyacrylamide gel at 10° C., 800 V, 50 mA, and 30 W for 3 hours in 1×TBE (90 mM Tris borate, 2 mM EDTA, pH 8.3) using an ALF-Express™ DNA-sequencer (Amersham Pharmacia Biotech, Uppsala, Sweden). The temperature was regulated by an external thermostat ALFexpress II Cooler system (Amersham Pharmacia Biotech, Uppsala, Sweden).

Specificity of the DNA-protein complex was confirmed by the addition of a 50-fold excess of either unlabelled specific competitor (SC, specific probe without the Cy5 label) or unlabeled non-specific competitor (UC). Signals were analyzed by ALFwin Fragment Analiser 1.03 (Amersham Pharmacia Biotech, Sweden) and presented as arbitrary units corresponding to area under curve (AUC).

Statistical Analysis

Comparison of the survival curves was performed using the Logrank test. In all cases, p values lower than 0.05 were considered as statistically significant.

### REFERENCES

1. Onyeama H P, Oehme F W: A literature review of paraquat toxicity. Vet Hum Toxicol 1984, 26:494-502.
2. Braithwaite R A: Emergency analysis of paraquat in biological fluids. Hum Toxicol 1987, 6:91-93.
3. Hart T B, Nevitt A, Whitehead A: A new statistical approach to the prognostic significance of plasma concentrations. Lancet 1984, 2:1222-1223.
4. Proudfoot A T, Stewart M S, Levitt T, Widdop B: Paraquat poisoning: significance of plasma-paraquat concentrations. Lancet 1979, 18:330-332.
5. Scherrmann J M, Houze P, Bismuth C, Bourdon R: Prognostic value of plasma and urine paraquat concentration. Hum Toxicol 1987, 6:91-93.
6. Dinsdale D, Preston S G, Nemery B: Effects of injury on [3H]putrescine uptake by types I and II cells in rat lung slices. Exp Mol Pathol 1991, 54:218-229.
7. Nemery B, Smith L L, Aldridge W N: Putrescine and 5-hydroxytryptamine accumulation in rat lung slices: cellular localization and responses to cell-specific lung injury. Toxicol Appl Pharmacol 1987, 91:107-120.
8. Rannels D E, Kameji R, Pegg A E, Rannels S R: Spermidine uptake by type II pneumocytes: interactions of amine uptake pathways. Am J Physiol Lung Cell Mol Physiol 1989, 257:L346-L353.
9. Rannels D E, Pegg A E, Clark R S, Addison J L: Interaction of paraquat and amine uptake by rat lungs perfused in situ. Am J Physiol Endocrinol Metab 1985, 249:E506-E513.
10. Smith L L: The identification of an accumulation system for diamines and polyamines into the lung and its relevance to paraquat toxicity. Arch Toxicol Suppl 1982, 5:1-14.
11. Clejan L, Cederbaum A I: Synergistic interaction between NADPH-cytochrome P-450 reductase, paraquat and iron in the generation of active oxygen radicals. Biochem Pharmacol 1989, 38:1779-1786.
12. Fernandez Y, Subirade I, Anglade F, Periquet A, Mitjavila S: Microsomal membrane peroxidation by an Fe3+/paraquat system. Consequences of phenobarbital induction. Biol Trace Elem Res 1995, 47:9-15.
13. Fukushima T, Yamada K, Isobe A, Shiwaku K, Yamane Y: Mechanism of cytotoxicity of paraquat. I. NADH oxidation and paraquat radical formation via complex I. Exp Toxicol Pathol 1993, 45:345-349.
14. Yamada K, Fukushima T: Mechanism of cytotoxicity of paraquat. II. Organ specificity of paraquat-stimulated lipid peroxidation in the inner membrane of mitochondria. Exp Toxicol Pathol 1993, 45:375-380.
15. Bus J S, Aust S D, Gibson J E: Superoxide- and singlet oxygen-catalyzed lipid peroxidation as a possible mechanism for paraquat (methyl viologen) toxicity. Biochem Biophys Res Commun 1974, 58:749-755.
16. Dicker E, Cederbaum A I: NADH-dependent generation of reactive oxygen species by microsomes in the presence of iron and redox cycling agents. Biochem Pharmacol 1991, 42:529-535.
17. Bus J S, Aust S D, Gibson J E: Lipid peroxidation: a possible mechanism for paraquat toxicity. Res Commun Chem Pathol Pharmacol 1975, 11:31-38.
18. Burk R F, Lawrence R A, Lane J M: Liver necrosis and lipid peroxidation in the rat as result of paraquat and diquat administration. Effect of selenium deficiency. J Clin Invest 1980, 65:1024-1031.
19. Bateman D N: Pharmacological treatments of paraquat poisoning. Hum Toxicol 1987, 6:57-62.
20. Bismuth C, Garnier R, Dally S, Fournier P E, Scherrmann J M: Prognosis and treatment of paraquat poisoning. A review of 28 cases. J Toxicol Clin Toxicol 1982:461-474.
21. Meredith T J, Vale J A: Treatment of paraquat poisoning in man: methods to prevent absorption. Hum Toxicol 1987, 6:49-55.
22. Okonek S, Hofmann A, Henningsen B: Efficacy of gut lavage, hemodialysis, and hemoperfusion in the therapy of paraquat or diquat intoxication. Arch Toxicol 1976, 36:43-51.
23. Vale J A, Meredith T J, Buckley B M: Paraquat poisoning: clinical features and immediate general management. Hum Toxicol 1987, 6:41-47.
24. Hershko C, Weatherall D J: Iron-chelating therapy. Crit Rev Clin Lab Sci 1988, 26:303-345.
25. Kohen R, Chevion M: Transition metals potentiate paraquat toxicity. Free Radic Res Commun 1985, 1:79-88.
26. Ranjbar A, Pasalar P, Sedighi A, Abdollahi M: Induction of oxidative stress in paraquat formulating workers. Toxicol Lett 2002, 131:191-194.
27. Evans P, Halliwell B: Micronutrients: oxidant/antioxidant status. Br J Nutr 2001, 85:S67-S74.
28. Sies H: Antioxidant activity in cells and organs. Am Rev Respir Dis 1987, 136:478-480.
29. Chen C J, Chin J E, Ueda K, Clark D P, Pastan I, Gottesman M M, Roninson I B: Internal duplication and homology with bacterial transport proteins in the mdr1 (P-glycoprotein) gene from multidrug-resistant human cells. Cell 1986 47(3):381-389.
30. Luce J M, Montgomery A B, Marks J D, Turner J, Metz C A, Murray J F: Ineffectiveness of high-dose methylprednisolone in preventing parenchymal lung injury and improving mortality in patients with septic shock. Am Rev Respir Dis 1988, 138:62-68.
31. Lin J L, Wei M C, Liu Y C: Pulse therapy with cyclophosphamide and methylprednisolone in patients with moderate to severe paraquat poisoning: a preliminary report. Thorax 1996, 51:661-663.
32. Chen C M, Lua A C: Lung toxicity of paraquat in the rat. J Toxicol Environ Health A 2000, 60:477-487.
33. Cho J H, Yang D K, Kim L, Ryu J S, Lee H L, Lim C M, Koh Y S: Inhaled nitric oxide improves the survival of the paraquat-injured rats. Vascul Pharmacol 2005, 42:171-178.
34. Roth G J, Calverley D C: Aspirin, platelets and thrombosis: theory and practice. Blood 1994, 83:885-898.
35. Jack D B: One hundred years of aspirin. Lancet 1997, 350:437-439.
36. Higgs G A, Salmon J A, Henderson B, Vane J R: Pharmacokinetics of aspirin and salicylate in relation to inhibition of arachidonate cyclooxygenase and antiinflammatory activity. Proc Natl Acad Sci USA 1987, 84:1417-1420.
37. Preston S J, Arnold M H, Beller E M, Brooks P M, Buchanan W W: Comparative analgesic and anti-inflammatory properties of sodium salicylate and acetylsalicylic acid (aspirin) in rheumatoid arthritis. Br J Clin Pharmacol 1989, 27:607-611.
38. DeWitt D L, el-Harith E A, Kraemer S A, Andrews M J, Yao E F, Armstrong R L, Smith W L: The aspirin and heme-binding sites of ovine and murine prostaglandin endoperoxide synthases. J Biol Chem 1990, 265:5192-5198.
39. Vane J R: Inhibition of prostaglandin synthesis as a mechanism of action for aspirin-like drugs. Nat New Biol 1971, 231:232-235.
40. Needs C J, Brooks P M: Clinical pharmacokinetics of the salicylates. Clin Pharmacokinet 1985, 10:164-177.
41. Brune K: Biodistribution of salicylates: a clue to the understanding of some effects and side effects. Agents Actions Suppl 1977, 2:163-177.
42. Stricker S: Aus der Taube'schen Klinik. Über die Resultate der Behandlung der Polyarthritis rheumatica mit Salicylsäure. Berl Klin Wochenschr 1876, 8:99-103.
43. Amann R, Peskar B A: Anti-inflammatory effects of aspirin and sodium salicylate. Eur J Pharmacol 2002, 447:1-9.
44. Tsujii M, DuBois R N: Alterations in cellular adhesion and apoptosis in epithelial cells overexpressing prostaglandin endoperoxide synthase 2. Cell 1995, 83:493-501.
45. Schwenger P, Bellosta P, Vietor I, Basilico C, Skolnik E Y, Vilcek J: Sodium salicylate induces apoptosis via p38 mitogen-activated protein kinase but inhibits tumor necrosis factor-induced c-Jun N-terminal kinase/stress-activated protein kinase activation. Proc Natl Acad Sci USA 1997, 94:2869-2873.
46. Yamamoto Y, Yin M J, Lin K M, Gaynor R B: Sulindac inhibits activation of the NF-kappaB pathway. J Biol Chem 1999, 274:27307-27314.
47. He T C, Chan T A, Vogelstein B, Kinzler K W: PPARdelta is an APC-regulated target of nonsteroidal anti-inflammatory drugs. Cell 1999, 99:335-345.
48. Jaarsveld Hv, Kuyl J M, Zyl G Fv, Barnard H C: Salicylate in the perfusate during ischemia/reperfusion prevented mitochondrial injury. Res Commun Mol Pathol Pharmacol 1994, 86:287-295.
49. Ghosh S, May M J, Kopp E B: NF-kappa B and Rel proteins: evolutionarily conserved mediators of immune responses. Annu Rev Immunol 1998, 16:225-260.
50. Whiteside S T, Israel A: I kappa B proteins: structure, function and regulation. Semin Cancer Biol 1997, 8:75-82.
51. Ghosh S, Karin M: Missing pieces in the NF-kappaB puzzle. Cell 2002, 109:S81-S96.
52. Bayon Y, Alonso A, Crespo M S: 4-trifluoromethyl derivatives of salicylate, triflusal and its main metabolite 2-hydroxy-4-trifluoromethylbenzoic acid, are potent inhibitors of nuclear factor kappaB activation. Br J Pharmacol 1999, 126:1359-1366.
53. Grilli M, Pizzi M, MMemo, Spano P: Neuroprotection by aspirin and sodium salicylate through blockade of NF-kappaB activation. Science 1996, 274:1383-1385.
54. Kopp E, Ghosh S: Inhibition of NF-kappa B by sodium salicylate and aspirin. Science 1994, 265:956-959.
55. Pierce J W, Read M A, Ding H, Luscinskas F W, Collins T: Salicylates inhibit I kappa B-alpha phosphorylation, endothelial-leukocyte adhesion molecule expression, and neutrophil transmigration. J Immunol 1996, 156:3961-3969.
56. Huang C J, Tsai P S, Lu Y T, Cheng C R, Stevens B R, Skimming J W, Pan W H: NF-kappaB involvement in the induction of high affinity CAT-2 in lipopolysaccharide-stimulated rat lungs. Acta Anaesthesiol Scand 2004, 48:992-1002.
57. Yang C H, Tsai P S, Lee J J, Huang C H, Huang C J: NF-kappaB inhibitors stabilize the mRNA of high-affinity type-2 cationic amino acid transporter in LPS-stimulated rat liver. Acta Anaesthesiol Scand 2005, 49:468-476.
58. Lowry O H N, Rosebrough N J, Farr A L, Randall R J: Protein measurement with Folin phenol reagent. J Biol Chem 1951, 193:265-275.
59. Ruscher K, Reuter M, Kupper D, Trendelenburg G, Dirnagl U, Meisel A: A fluorescence based non-radioactive electrophoretic mobility shift assay. J Biotechnol 2000, 78:163-170.

1. Utilization of the salicylate and its precursors or derivatives characterized in that they are to be applied in the treatment of paraquat mammal's intoxications trough the decrease of the pulmonary lesions resulting from the toxicity of this xenobiotic to the cells, organs and to the mammal's organisms.

2. The utilization of the salicylate and its precursors or derivatives accordingly to claim 1, characterized in that they increase the survival of the paraquat intoxicated animals to 100% by inhibiting the NF-kB activation, when administered in dosages comprised within 20 and 200 mg/Kg.

3. The utilization of the salicylate and its precursors or derivatives accordingly to claim 2, characterized in that they decrease the acute and chronic effects caused by paraquat, turning a dose susceptible to cause multiorganic failure and thus potentially fatal, into a non toxic dose and in that they are administered in multiple daily dosages comprised within 20 and 100 mg/Kg of body weight, two, three or four times daily.
